(19) United States
(12) Patent Application Publication
(10) Pub. No.: US 2007/0150052 A1
(43) Pub. Date: Jun. 28, 2007

(54) MANUFACTURE OF PROSTHETIC TISSUE HEART COMPONENTS

(76) Inventor: Albert N. Santilli, Pepper Pike, OH (US)

Correspondence Address: RANKIN, HILL, PORTER & CLARK, LLP, 925 EUCLID AVENUE, SUITE 700, CLEVELAND, OH, 44115-1405, US

(21) Appl. No.: 11/614,238

(22) Filed: Dec. 21, 2006

Related U.S. Application Data
(60) Provisional application No. 60/753,569, filed on Dec. 23, 2005.

(51) Int. Cl. A61F 2/24

(52) U.S. Cl. 623/2.11; 623/904; 600/425

(57) ABSTRACT

A technique for manufacturing a prosthetic tissue heart component involves performing a non-invasive scanning operation on a patient's heart, preferably by CT angiography. Images of the specific dimensions of the heart component to be replaced are generated. The images generated by the scanning operation are used to manufacture the replacement heart component. The invention permits a custom-fit replacement heart component to be installed in a patient. Use of the invention also avoids the need to manufacture or fit the replacement heart component during the course of a surgical procedure.

### REFERENCE TO PROVISIONAL APPLICATION

This application claims priority from U.S. provisional application Ser. No. 60/753,569, filed Dec. 23, 2005 by Albert N. Santilli, the disclosure of which is incorporated herein by reference.

### BACKGROUND OF THE INVENTION

1. Field of the Invention

The present invention relates to prosthetic heart components made of biological tissue and, more particularly, to tissue heart components, particularly valves, that are precisely sized for an optimized fit.

2. Description of the Prior Art

Cardiovascular surgeons typically use one of two types of prosthetic heart valves to replace a patient's diseased or damaged heart valve. The two types of prosthetic heart valves are mechanical valves and valves made of biological tissue. Mechanical valves are superior in durability but have a tendency to cause blot clotting on their surfaces and require the patient to rely on taking anticoagulants for the rest of the patient's life.

While prosthetic heart valves made of biological tissue are not believed to be as durable as mechanical valves due to eventual calcification, they do not tend to cause blood clotting, thereby avoiding the need for the patient to take anticoagulants. Typical biological heart valves include porcine or bovine heart valves—that is, heart valves taken from a pig or a cow. Unfortunately, existing methods for implanting tissue valves into a patient lack means to provide a precise fit. Surgeons must settle for choosing the closest size from among tissue valves of varying predetermined sizes; the surgeon then must do his or her best to form or adapt the valve to fit the area in the patient where the prosthetic valve will be placed.

Existing methods for sizing a prosthetic tissue valve include using an obturator to determine the size of the valve that is needed and a template to guide the surgeon in cutting the valve. These obturators and templates often include a set number of standardized sizes from which to choose. See, for example, U.S. Pat. No. 5,326,371 and U.S. Pat. No. 6,342,069, the disclosures of which are incorporated herein by reference. Not only are the disclosed techniques limited to certain pre-determined sizes of valves, but they also require that the valve be assembled on an emergency basis while the patient is on the operating table. For example, the '371 patent sets forth a standard of assembling an autogenous tissue valve in 10 minutes or less.

A similar situation exists with respect to sizing other heart components that may need to be replaced, such as aortic and pulmonic valve conduits, mitral valves and mitral valve repairs, annuloplasty rings, internal mammary arteries, and pericardial patches. Desirably a technique would be available that would enable custom-fit heart components such as valves to be manufactured. Preferably, such components could be manufactured prior to the commencement of a surgical procedure such that no downtime would be required for component manufacture or fitting during the course of the surgical procedure itself.

### SUMMARY OF THE INVENTION

In response to the foregoing concerns, the present invention provides a new and improved technique for manufacturing replacement heart components such as valves prior to the commencement of a surgical procedure. The invention includes the steps of performing a non-invasive scanning operation on a patient's heart and generating images of the specific dimensions of a heart component to be replaced. Preferably, the scanning operation is computed or computerized (CT) angiography or equivalent or better measuring technology. The invention includes the step of using two-dimensional and/or three-dimensional images generated by the scanning operation to manufacture a prosthetic tissue heart valve or other necessary component to the patient's particular dimensions. The use of pre-surgical scanning and manufacture allows the surgeon to provide the patient with a custom-fit tissue heart valve or other component, while avoiding manufacturing or fitting downtime during the course of the surgical procedure itself.

The foregoing, and other features and advantages of the invention, will be apparent from reviewing the accompanying description and claims.

### DESCRIPTION OF THE PREFERRED EMBODIMENT

The present invention includes a method for manufacturing and custom-fitting a heart component such as a prosthetic tissue heart valve precisely to the patient's particular dimensions. The tissue for the heart valve preferably is porcine or bovine, but may comprise other types of biological tissue, including but not limited to human tissue. Conceivably autologous tissue could be used, especially if it could be harvested prior to commencement of the surgical procedure. Suitable tissue heart components such as aortic valves, aortic valve conduits, mitral valves, annuloplasty rings, internal mammary arteries, pulmonic valve conduits, and pericardial patches are commercially available from Shelhigh, Inc., 650 Liberty Avenue, Union, N.J. 07083 under the trademark NO-REACT.

The method includes performing a non-invasive scanning operation, preferably by using computed or computerized (CT) angiography or equivalent or better technology on the patient to obtain a computer-generated image (CT angiogram) of the area of the heart in which the prosthetic component is to be placed. The CT angiogram may be performed in any manner known in the art, but typically includes the following steps: introducing a contrast dye into the area of the heart in which a prosthetic tissue valve is to be placed; scanning the latter area with a CT scanner; generating two-dimensional and/or three-dimensional images of the affected area; and viewing computer-generated images of the scanned area.

Image types that the computer may generate preferably are three-dimensional images, but two-dimensional images may be acceptable. Suitable software for creating the requisite images is commercially available from True Life Anatomy Pty Ltd., 128 Hindley Street, Adelaide, Australia. Information concerning the software is available at the company's web site, www.truelifeanatomy.com, and at the web site of the company's distributor, RuBaMAS, www.rubamas.com, the disclosures of which are incorporated herein by reference.

As set forth in more detail in the referenced web sites, the True Life Anatomy software includes a TLA generator, a TLA viewer, and a TLA animator. The TLA generator imports CT (or MRI)-scanned two-dimensional slice data and creates three-dimensional models that are saved as TLA files. TLA files contain both the three-dimensional model created by the TLA generator software as well as the CT slice data in a compressed form. The three-dimensional model can be sent to a clinician for viewing on the TLA viewer that requires less computing power. This three-dimensional image data also can be transmitted by network or broadband connection.

The TLA generator reads in raw CT data and converts it to surface models and generates the .tla file format. The TLA generator allows for individual control of creating separated segments, or automatic functions for best guess separation. It also allows separation of component parts of the object and can delete parts of the objects, as well as measure distances and angles. The TLA viewer plays back .tla files and saves to .jpg format or prints reports. The viewer can hide objects or segments, color objects or segments, and tag objects or segments with descriptors or notes. The TLA animator creates native .tlm animation files that can be viewed on the TLA viewer by reading in .tla files and generating animations. These can be sequential or generated using a virtual camera in three-dimensional space.

The CT scanner may be a conventional CT angiogram scanner. More desirably, the CT angiogram scanner may be a multislice scanner that provides clearer pictures than that of some other scanners. The multislice scanner also provides images much more efficiently and faster than other scanners. One such suitable multislice CT scanner is commercially available from Siemens AG, Medical Solutions, Erlangen, Germany under the mark SOMATOM Sensation 64. The multislice scanner in question makes 64 slices per rotation with an isometric resolution of less than 0.4 mm. The action of the heart virtually can be stopped as the scanner can take over 190 slices per second. The multislice scanner in question also is available with diagnostic software that produces three-dimensional images.

The present invention further includes using the computer-generated images to precisely custom-fit a tissue heart component such as a valve to the area in which the component is to be placed in the patient. If a valve is being replaced, the valve may be any type of heart valve, including but not limited to the aortic valve and the mitral valve, and repairs to the soft tissue around the valves.

The CT angiogram and diagnostic software provide very precise information as to the size and location of the structures within the patient's heart. This information is provided in an efficient, non-invasive, and relatively economical manner. The use of the CT angiogram and diagnostic software to provide precise dimensions represents a significant advantage in the art of manufacturing tissue heart components because such components now can be manufactured for the specific patient upon which the surgery is about to be performed. Moreover, since the components can be manufactured prior to the commencement of the surgical procedure, no downtime is required for component manufacture or fitting during the course of the surgical procedure itself.

Although the invention has been described in its preferred form with a certain degree of particularity, it will be understood that the present disclosure of the preferred embodiment has been made only by way of example, and that various changes may be resorted to without departing from the true spirit and scope of the invention as hereinafter claimed. It is intended that the patent shall cover, by suitable expression in the appended claims, whatever degree of patentable novelty exists in the invention disclosed.

What is claimed is:

1. A method of manufacturing a replacement prosthetic tissue heart component for a patient, comprising the steps of:
scanning the patient's heart non-invasively;
generating images of a heart component to be replaced by using the results of the non-invasive scan;
determining the specific dimensions of the heart component to be replaced from the images generated by the non-invasive scan; and
manufacturing the replacement heart component using the images of the specific dimensions of the heart component to be replaced.

2. The method of claim 1, wherein the step of scanning the patient's heart non-invasively is accomplished by CT angiography.

3. The method of claim 2, wherein the CT angiography is performed by using a multislice scanner.

4. The method of claim 1, wherein the images generated by the scanning operation are two-dimensional and three-dimensional images.

5. The method of claim 1, wherein the step of scanning is accomplished by CT angiography and the images generated by the scanning operation are generated by computer software.

6. The method of claim 1, further comprising the steps of:
performing a surgical procedure on the patient to remove the heart component to be replaced; and
installing the replacement heart component.

7. The method of claim 6, wherein the steps of scanning the patient's heart non-invasively, generating images of a heart component to be replaced by using the results of the non-invasive scan, determining the specific dimensions of the heart component to be replaced from the images generated by the non-invasive scan, and manufacturing the replacement heart component using the images of the specific dimensions of the heart component to be replaced are performed prior to the step of performing a surgical procedure on the patient to remove the heart component to be replaced.

8. The method of claim 1, wherein the heart component to be replaced is selected from the group consisting of aortic valves, aortic valve conduits, mitral valves, annuloplasty rings, internal mammary arteries, pulmonic valve conduits, and pericardial patches.

9. The method of claim 1, wherein the heart component is porcine, bovine, or human tissue.

10. A method of replacing a defective heart component of a patient, comprising the steps of:
scanning the patient's heart non-invasively using a multislice CT scanner;
generating two-dimensional and three-dimensional images of the defective heart component by using computer software;
determining the specific dimensions of the defective heart component from the images generated by the non-invasive scan;
manufacturing a replacement heart component from porcine, bovine, or human tissue using the images of the specific dimensions of the defective heart component;
performing a surgical procedure on the patient to remove the defective heart component; and
installing the replacement heart component.

11. The method of claim 10, wherein the replacement heart component is manufactured prior to conducting the step of performing a surgical procedure on the patient to remove the defective heart component.

12. The method of claim 10, wherein the replacement heart component is selected from the group consisting of aortic valves, aortic valve conduits, mitral valves, annuloplasty rings, internal mammary arteries, pulmonic valve conduits, and pericardial patches.

13. A replacement heart component for a patient's heart made by the steps of:
scanning the patient's heart non-invasively;
generating images of a heart component to be replaced by using the results of the non-invasive scan;
determining the specific dimensions of the heart component to be replaced from the images generated by the non-invasive scan; and
manufacturing the replacement heart component using the images of the specific dimensions of the heart component to be replaced.

14. The replacement heart component of claim 13, wherein the non-invasive scan is CT angiography.

15. The replacement heart component of claim 14, wherein the CT angiography is performed by using a multislice scanner.

16. The replacement heart component of claim 13, wherein the images generated by the scanning operation are two-dimensional and three-dimensional images.

17. The replacement heart component of claim 13, wherein the non-invasive scan is CT angiography and the images are generated by computer software.

18. The replacement heart component of claim 13, wherein the heart component to be replaced is selected from the group consisting of aortic valves, aortic valve conduits, mitral valves, annuloplasty rings, internal mammary arteries, pulmonic valve conduits, and pericardial patches.

19. The replacement heart component of claim 13, wherein the heart component is porcine, bovine, or human tissue.
